(19) United States
(12) Patent Application Publication
(10) Pub. No.: US 2008/0050816 A1
(43) Pub. Date: Feb. 28, 2008

(54) Cell culture chip with a III-V nitride compound adhesion layer and applied thereof

(75) Inventor: Chi-Ruei Chen, Changhua City (TW)

Correspondence Address: BIRCH STEWART KOLASCH & BIRCH, PO BOX 747, FALLS CHURCH, VA, 22040-0747, US

(73) Assignee: ZONG-SIAN YAN

(21) Appl. No.: 11/892,632

(22) Filed: Aug. 24, 2007

(30) Foreign Application Priority Data
Aug. 25, 2006 (TW) 095131389

(51) Int. Cl. C12N 5/06; C12M 1/00

(52) U.S. Cl. 435/402; 435/288.3; 435/307.1

(57) ABSTRACT

The present invention discloses a cell culture chip, which comprising a cell adhesion layer with a III-V nitride compound. The cell adhesion layer according to the invention can improve the adhesion effect between the cultured cell and the cell adhesion, enhance the cell growth and differentiation, and reduce the cell death rate. The present invention further discloses an application of the cell adhesion.

### BACKGROUND OF THE INVENTION

The invention relates a cell culture chip, which concerns to a cell adhesion layer with a III-V nitride compound.

In recent years, it has already caused attention about the neuron chip for bonding electron and the biosystem can stimulate the neuron cell or collects the neuron to send out signal. Mainly is because of the neuron chip potential application in the fields of medicine, for instance: This neuron chip may develop to take one medicine sieves way for the drugs manufacture industry which can find out the precursor faster. Or combine the cell with electric silicon circuit which may create the artificial limb of neuron, it can treat the neuron unusual. In technical viewpoint, the neuron chip development which provides the cell tightly to suitable chip surface department is most important condition. At present used in chip of the neuron system, mostly people use silicon sill as the base material. However, the silicon has very low biological compatibility to the neuron chip, in order to promote the cell attaching, growing and maintaining, often coating biological activity member on the surface of silicon, for example: poly-D-Lysine, collogen, fibronectin or laminin. But this kind of biological activity member generally absorb and distribute on the surface of the silicon sill with physics, the biological activity member is extremely apt to shed in the washing step. The general silicon surface often coating the biological activity member is poly-D-Lysine, as the growth of time, it may shed or broken on the silicon surface. Some research papers pointed out, when the concentration of poly-D-Lysine shredded part is excessively high, the phenomenon that will cause cells cytotoxity is produced.

### SUMMARY OF THE INVENTION

In accordance with the present invention, a cell culture chip comprising a cell adhesion layer with a III-V nitride compound.

In one embodiment, a device of cell culture chip which is include a base plate and a cell adhesion layer with III-V group nitride compound. The cell culture chip needn't coat biological activity member like Poly-D-Lysine on the surface. it can provide a place for culture cell to adhere and growth.

The purpose of the invention, a method of cell culture wherein culture cell efficiently, and reduce the death rate of wanted culture cell.

Another purpose of the invention, providing a artificial neural chip combines with the cell culture chip which can be used to stimulate or receive the electronic signal from neuron cell, set up and contact information of the living body cell directly, in order to study the spread of the brain function and neural signal.

In order to reach the above-mentioned main purpose, a cell culture chip of the present invention include a base plate; and a cell adhesion layer which is formed on surface of the base plate which is provided a place for culture cell to adhere and growth; wherein the cell adhesion layer is a III-V group nitride film.

The cell culture chip of invention comprises a buffer layer among the base plate and the cell adhesion layer. The buffer layer may be nitrogen aluminium or nitrogen gallium.

The cell culture chip of invention wherein the base plate is consisted from each of sapphire, SiC, spinel and GaAs group; the III-V group nitride film is consisted from one of nitrogen aluminium, nitrogen gallium and nitrogen indium group; the III-V group nitride film is grown on the surface of base plate by metalorganic chemical vapor deposition, molecular beam epitaxy and hydride vapor phase epitaxy.

The present invention also discloses another method for cell culture, comprising: providing a cultivation container; providing a cell adhesion layer which set in the cultivation container and the cell adhesion layer is one of III-V group nitride compound; providing a culture put in the cultivation container; and inoculating a wants cell on surface of the cell adhesion layer. The III-V group nitride compound is consisted from each of nitrogen aluminium, nitrogen gallium and nitrogen indium group.

The present invention also discloses another artificial neural chip combines with the cell culture chip, comprising: a base plate; a circuit layer which is formed on the surface of the base plate; and a cell adhersion layer which is formed on the surface of the circuit layer; providing a place for cell to adhere and growth wherein the cell adhesion layer is a III-V group nitride film.

The present invention of artificial neural chip, where, the base plate is base plate of glass or silicon base plate; every sides of this circuit and the cell adhersion layer have numbers of electrode, and numbers of wire link with electrode; wherein the electrode is selected from one of gold, silver, rhenium, ruthenium, rhodium, Pd, silver, osmium and iridium. The wire is comprising from one of indium oxide, tin oxide, cadmium tin oxide, antimony, chromium, silver, copper of the zinc oxide, etc. or metal alloy.

A implementation method of the present invention, cell culture with III-V nitride compound which is on the cell culture chip surface of cell adhesion layer, Compares with other culture medium materials, without coating biological activity member like poly-D-Lysine, it also strengthens the cell and the cell adhesion layer efficiently, it also can promote the growth and division of the cell, reduce cell's death rate. The cell culture chip can be used in culture of nerve cell, neural stem cell, artificial neural cell, muscle cell and myocardium cell. Another implementation method of the present invention, artificial neural chip with III-V nitride compound of cell adhesion layer, it can be use to provide a place for cell attach and growth, and by inputting the electric current to the cell, stimulate the cell activity and uses to observe the signal change among cells.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The foregoing aspects and many of the attendant advantages of this invention will become more readily appreciated as the same becomes better understood by reference to the following detailed description, when taken in conjunction with the accompanying drawings, wherein:

FIG. 1 is the cell culture chip of the invention.

FIG. 2 is the horn neuron adhere on base plate of cell adhesion layer after 6 days on different cell, (A) u-type GaN, (B) n-type GaN, (C) p-type GaN, (D) Silicon, and (E) TCPS (without putting into any chip of culturing the polyethylene plate, as controlling the group).

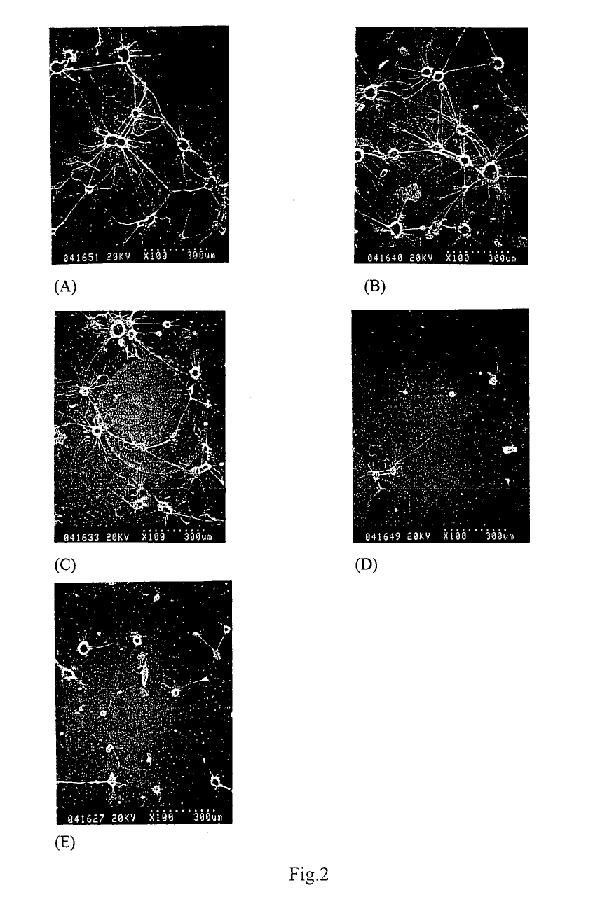

FIG. 3 is the relatively remaining rate of the neuron.

FIG. 4 is the cerebellum granule neurons growth after 35 days in cell culture chip with different cell adhesion layer, (A) GaN, (B) Poly-Lysine, and (C) TCPS (as control group).

FIG. 5 is the growth situation 30 days later in the nitrogen gallium chip for the neural stem cell.

FIG. 6 is the phosphoprotein degree of Akt and ERK of the he PC12 cell is cultured in different cell adhesion layer material

FIG. 7 is the artificial neural chip of the invention.

FIG. 8 is the cortical stem cells were cultured for 21 days on (a) n-type GaN, (b) PDL and (c) TCPS. Double staining for nestin expressing neural stem cell (red) and MAP-2 expressing astrocyte (green). (scale bar=100 μm)

FIG. 9 is the cortical stem cells were cultured for 21 days on (a) n-type GaN and (b) PDL (c) TCPS. Double staining for nestin (red) and GFAP (green). (scale bar=100 μm)

FIG. 10 is the cortical stem cells were cultured for 21 days on (a) n-type GaN and (b) PDL (c). Double staining for synapsin1 expressing neuron (red) and MAP-2 (green). (scale bar=100 μm)

### DESCRIPTION OF THE PREFERRED EMBODIMENT

The following is a description of the present invention. The invention will firstly be described with reference to one exemplary structure. Some variations will then be described as well as advantages of the present invention. A preferred method of fabrication will then be discussed.

### Experimental 1

### Preparation of Cell Culture Chip

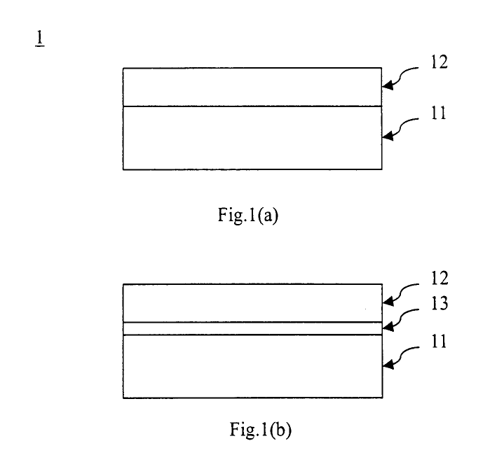

As shown in FIG. 1, the present invention of cell culture chip (1), include a base plate (11) and a cell adhesion layer (12) with III-V group nitride film which is formed on surface of the base plate providing a place for culture cell to adhere and growth. Among them, this base plate select from each of sapphire, SiC, spinel and GaAs group. The III-V group nitride film (12) is consisted from one of nitrogen aluminium, nitrogen gallium and nitrogen indium group, and grown on the surface of base plate (11) by metalorganic chemical vapor deposition, molecular beam epitaxy and hydride vapor phase epitaxy. In addition, the cell culture chip (1) containes a buffer layer (13) which is put between the base plate (11) and the cell adhesion layer (12). Imporving the problem of lattice constant and thermal-expansion coefficient among the cell adhesion layer with III-V group nitride film (12) and the base plate (11). Among them, the buffer layer (13) is ach of nitrogen aluminium, and nitrogen gallium.

For example, the present invention of cell culture chip (1), wherein the nitrogen gallium film (12) grows on the base plate of sapphire (11) by metalorganic chemical vapor deposition. Before epitaxy, the base plate of sapphire (11) need to carry on the surface cleaning process in advance, and get rid of Superficial oil and oxidizing layer on this base plate (11). In this example, using separate-flow horizontal reactor to prepare the cell culture chip of nitrogen gallium. (Kuwan N, Tsukamoto K, Taki W, Horibuchi K. Gradual tilting of crystallographic orientation and configuration of dislocations in GaN selectively grown by vapour phase epitaxy methods. J Electron Microsc (Tokyo). 2000; 49:331-8.).

The main reactant source separate by the quartz slate separation, which is introduced the flow rate of 60 mmol/min in trimethylgallium˜TMGa and 5000 cc/min in NH3 flowing into the upper and the bottom streams, as the sources of the nitrogen gallium. Firstly, the base plate of sapphire (11) temperature is set at 525° C., and it will grow up to 0˜1000 Å thickness of gallium buffer layer; then, the base plate (11) improves to 100° C., a nitrogen gallium epitaxy layer was form on nitrogen gallium buffer layer. Make out a cell culture chip (1) of cell adhesion layer with U-type nitrogen gallium of cell adhesion layer (12)(U-type GaN cell culture chip). When the temperature is up to 1130° C., the nitrogen gallium film (12) of the U-type GaN cell culture chip (1) will enter SiH4 gas, and produce silicons which is mixing into the nitrogen gallium film (12). The silicomethane of flow rate is between 0.43˜0.65 nM/min, and we will get a N-type GaN cell culture chip. As for the P-type GaN cell culture chip, we can enter (C5H5)2Mg.Cp2Mg gas on the surface of U-type GaN cell culture chip, and produces magnesium and mixes into the surface of nitrogen gallium film (12).

The present invention of cell culture chip (1) can be suitable for culture cell which may be nerve cell, neural stem cell, artificial neural cell, muscle cell and myocardium cell.

### Experimental 2

### Assessment of Cell Culture

Deal with Cell Culture Chip

Before culturing of cell, we must deal with the cell culture chip (1). First of all, chip is cut into the size of 1 cm2, and wash with 70% of the alcohol; Then carry on sterilization with autoclave, and wash with large amount of phosphoric acid (PBS).

After sterilization, put the cell culture chip (1) into the 24 holes of polyethylene plate (TCPS plate, buy from corning, New York of U.S.A.).

Nerve Cell Culture

Cerebellar granule neurons were prepared from 7-day-old Wistar rats. Briefly, neurons were dissociated from freshly dissected cerebelli by mechanical disruption in the presence of trypsin and DNase. Neurons were added to the 24 holes of culture wells at a density of 10×106 cells/well in basal Eagle medium (BME; Gibco) supplemented with 10% fetal calf serum (FCS; Gibco), 25 mm KCl, penicillin G (100 IU/ml) and streptomycin (100 mg/ml). Cultures were maintained at 37° C. in a humidified atmosphere of 95% air/5% CO2. Cytosine arabinoside (10 μm) was added to the culture medium 1 day after plating to prevent replication of non-neuronal cells. (Autoradiographic localization and depolarization-induced release of acidic amino acids in differentiating cerebellar granule neurons J. Biomed Mater Res 2000; 52: 748-53.)

Assessment of Neuron Morphology

The present invention, vaccination neuron is set into different cell adhesion layer of cell culture chip (1) which is put in 24 holes of medium plates. The cell adhesion layer (12) is each of U-type GaN, P-type GaN, N-type GaN, U-type Si and TCPS (as control group). For morphological observation, the cells adhering to the membrane were washed with PBS and then fixed with 2.5% glutaraldehyde in PBS for 1 h at 4° C. Subsequently, the cells were postfixed for 1 h in 1% osmium tetroxide at 25° C. Afterthorough washing with PBS, the specimens were dehydrated by graded ethanol

changes, critical point dried and examined by metallography microscopy (Olympus BX51M, Japan).

Table 1 is cerebellum granule neurons culture in medium and the growth of axon and dendrite after 3, 6, 12 days. As shown in the table, after 3 days, the cerebellum granule neurons presents good adhesion situation on the surface of the nitrogen gallium cell culture chip (1), we cam see the growth of axon and dendrite. When the cell culture on silicon chip or in the TCPS directly, the neuron presents a phenomenon of gathering, and without producing the high density neural net on the surface of nitrogen gallium chip. When the cerebellum granule neurons is cultured after 6 days (As shown in pic. 2), the cerebellum granule neurons presents numbers do not have obviously changed, but the number of axon and dendrite increases obviously. The cerebellum granule neurons and axon and dendrite which is cultured on the surface of silicon chip large decrease. When the cerebellum granule neurons is cultured after 12 days, the cerebellum granule neurons presents numbers do not have obviously changed, and slightly present the phenomenon gathered, the number of axon and dendrite has the tendency to reduce, this phenomenon may be because with the increase of culturing days, nutrition factors are insufficiently inside, the cell is inclined to assembling to achieve the goal of surviving each other. There are few cerebellum granule neurons cultured on silicon chip or in the TCPS directly.

TABLE1
The adhesion density of nerve cell and the growth of axon and dendrite

| | 3 days | | 6 days | | 12 days | |
|---|---|---|---|---|---|---|
| | | the growth of | | the growth of | | the growth of |
| | Cell | axon and | Cell | axon and | Cell | axon and |
| material type | adhesion | dendrite | adhesion | dendrite | adhesion | dendrite |
| U-type GaN | ***** | **** | ***** | ***** | ***** | **** |
| N-type GaN | ***** | **** | ***** | ***** | ***** | **** |
| P-type GaN | **** | *** | **** | **** | **** | *** |
| U-type Si | *** | ** | ** | * | * | * |
| TCPS | ** | ** | *** | *** | ** | ** |

***** more,
**** many,
*** ordinary,
** few,
* less

Assessment of Neuronal Survive

LDH assay was used to count cell survived on the GaN films. For the LDH assay, the cell culture chip (1) which is cultured in different cell adhesion layer (12) were incubated with 0.2 ml of Triton X-100 for 30 min at 37° C., in order to destroy the cell membrane. Then the neuron surgical which is released to LDH medium was evaluated, it can be the a indicator with remaining neuronal.

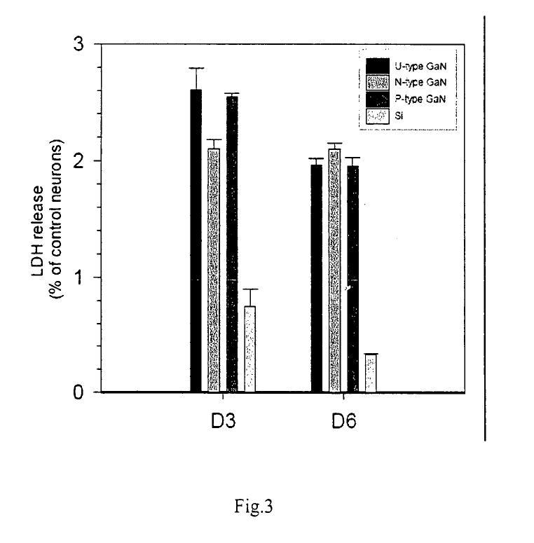

FIG. 3 shows the relative cell number 3 and 6 days after cell seeding, which was quantitatively determined from the ratio of the LDH released from survival cells attached to the films to that of cells attached to TCPS. The LDH assay relies on the ability of the viable cells, thus the LDH value obtained is directly proportional to the cell number on each film. Cells cultured on all GaN films show near to or more than 200% survival of cells relative to TCPS. However, only 74±15% and 33±0.7% cell survival were observed on the silicon film relative to TCPS after 3 and 6 days in culture. LDH assay confirmed qualitative observation of neuron morphologies. Thus, the effect of GaN and silicon films on the cultured neurons was noted, indicating that the GaN films may be used to culture neurons.

### Experimental 3

### Assessment of Culture Cell for Long Day

The Situation of Neuron Growth in Different Medium

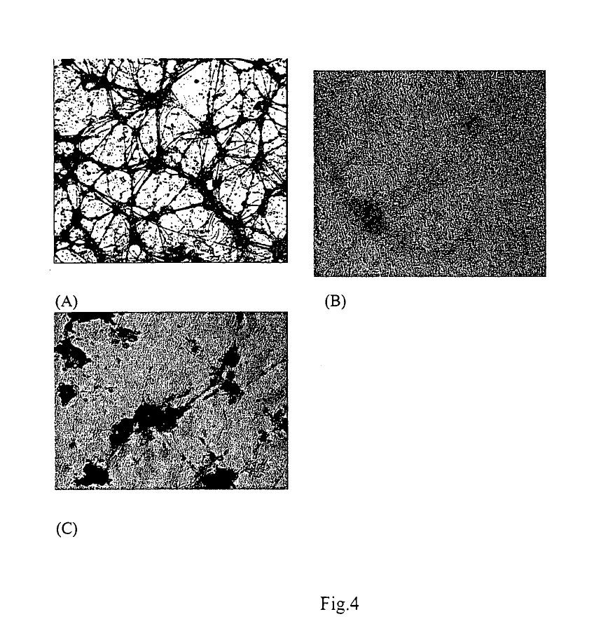

FIG. 4 shows the cerebellum granule neurons growth after 35 days in cell culture chip with different cell adhesion layer, (A) GaN, (B) Poly-Lysine (coating on TCPS), and (C) TCPS (without putting any material in the polyethylene plate as control group). The cultivation condition of cerebellum granule neurons are described as previously. It shows that the neuron culture in the cell adhesion layer with Gan, the cell number and the differentiation of axon and dendrite will be larger than the cell culture in the cell adhesion layer with Poly-Lysine and TCPS. This invention discloses a cell cultured chip (1) which is useful to cell survival, growth and differentiation.

Growth Situation of the Neural Stem Cell

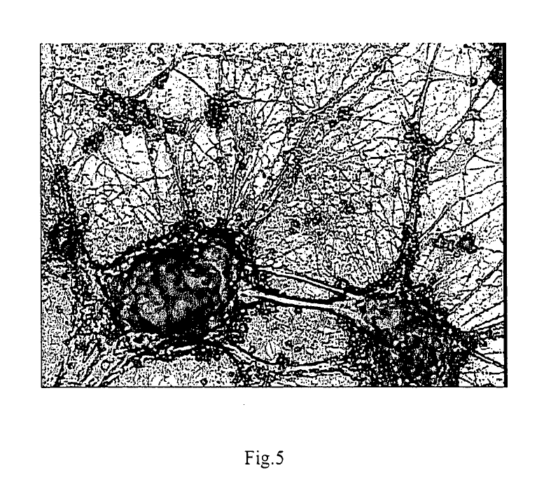

FIG. 5 shows the situation of neural stem cell growth in the cell culture chip (1) after 30 days. As shown in the picture, the neural stem cell obviously growth the neural network.

Assessment the Culture of Pheochromocytoma (PC12)

PC12 cell sample separates from big mouse's adrenal pheochromocytoma. First of all, the PC12 culture cell is including of 7.5% heat-inactivated fetal bovine serum, 7.5% horse serum, 2 mM HEPES buffer solution and 44 mM sodium bicarbonate in the mericlone bottle with the DMEM medium. Cultures are maintained at 37° C. in a humidified atmosphere of 95% air/5% CO2. Culture 3 days latter, PC12 cell move in TCPS medium plate with 24 holes which is cultured in a medium of DMEM/F12. After cultivation of 2 hours, 50 ng/ml NGF and 0.25% BSA put in the medium. After cultivation of 1 days, the PC12 cell is analyzed the phosphoprotein degree of serine-threonine kinase and extracellular signal-regulated kinase by Western blot.

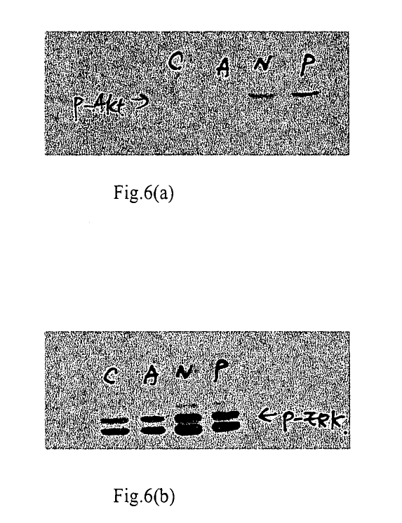

FIG. 6 shows the phosphoprotein degree of Akt and ERK of the he PC12 cell is cultured in different cell adhesion layer material; wherein the medium C is TCPS (as control group), A is Poly-D-Lysine coating on surface of holes N is U-type GaN, P is P-type GaN. In PC12 cell, as the phosphoprotein degree of Akt (P-Akt) is high, it can promote surviving, suppressing cells to wither and die of the cell. When the phosphoprotein degree of ERK (P-ERK) is high, it can promote the cell behaving and differentiation. As shown in figure, the phosphoprotein degree of Akt and ERK are larger than TCPS.

### Experimental 4

### Artificial Neural Chip

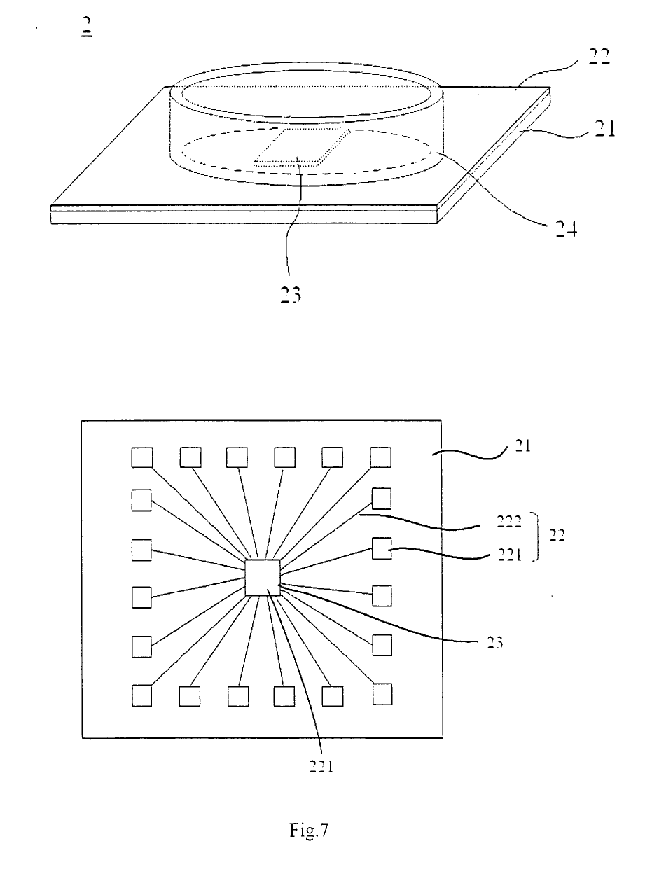

FIG. 7 shows a device of artificial neural chip (2) combines with a cell culture chip, comprising: a base plate (21); a circuit layer (22) which is formed on the surface of the base plate (21); and a cell adhersion layer (23) which is formed on the surface of the circuit layer (22) which is providing a place for cell to adhere and growth wherein every sides of this circuit layer (22) and cell adhersion layer (23) have numbers of electrode (221) which is linking by numbers of wires. The artificial neural chip (2) also can include a cell medium (24) which is set on the surface of circuit layer (22), and around the cell adhersion layer (23).

The present invention of the artificial neural chip (2) wherein the base plate (21) is glass or silicon base plate; the electrode (221) is selected from one of gold, silver, rhenium, ruthenium, rhodium, Pd, silver, osmium and iridium; numbers of wire can be each of indium oxide, tin oxide, cadmium tin oxide, antimony, chromium, silver, copper of the zinc oxide, etc. or metal alloy; the III-V group nitride film is consisted from one of nitrogen aluminium, nitrogen gallium and nitrogen indium group.

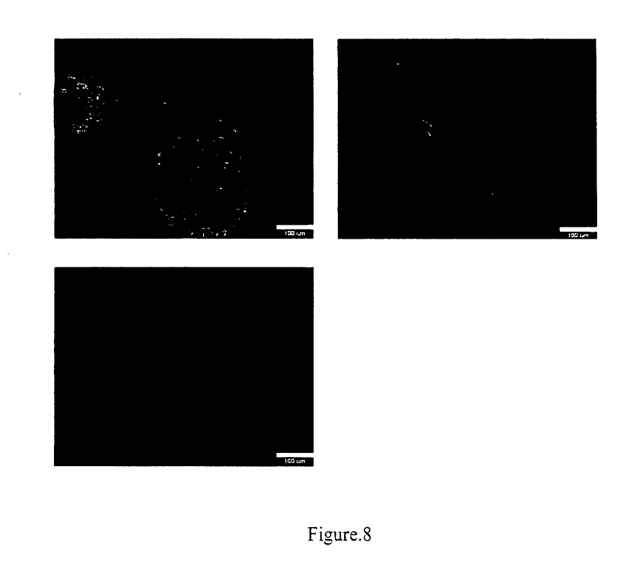

FIG. 8 shows the cortical stem cells were cultured for 21 days on (a) n-type GaN, (b) PDL and (c) TCPS. Double staining for nestin expressing neural stem cell (red) and MAP-2 expressing astrocyte (green). (scale bar=100 μm)

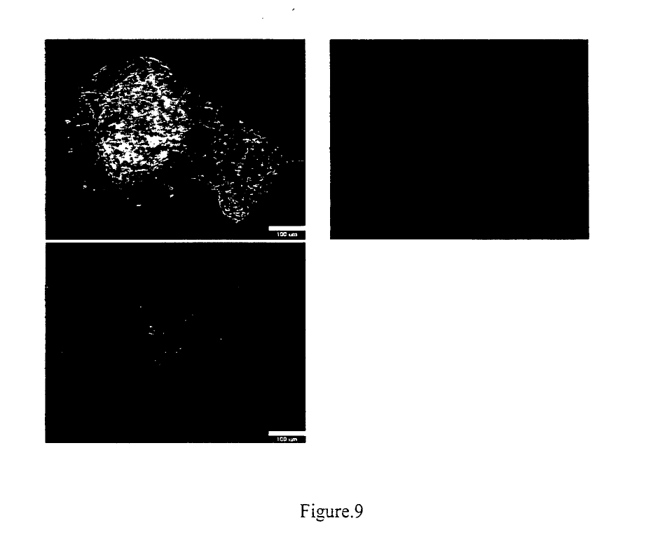

FIG. 9 shows the cortical stem cells were cultured for 21 days on (a) n-type GaN and (b) PDL (c) TCPS. Double staining for nestin (red) and GFAP (green). (scale bar=100 μm)

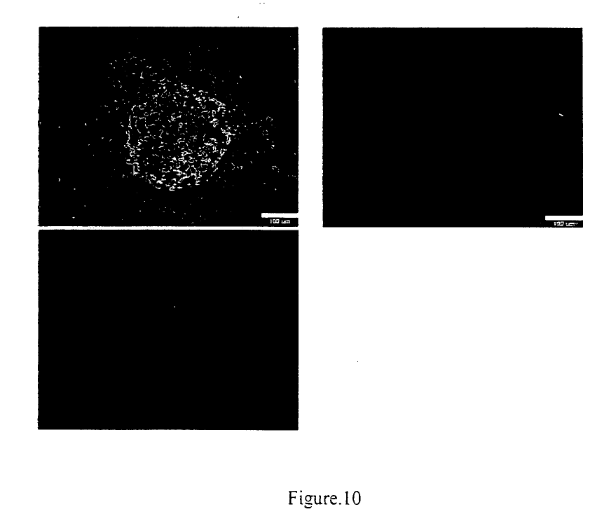

FIG. 10 shows the cortical stem cells were cultured for 21 days on (a) n-type GaN and (b) PDL (c). Double staining for synapsin1 expressing neuron (red) and MAP-2 (green). (scale bar=100 μm)

1. A device of cell culture chip, include:
a base plate; and
a cell adhesion layer which is formed on surface of the base plate;
providing a place for culture cell to adhere and growth;
wherein
the cell adhesion layer has a III-V group nitride film.

2. The device according to claim 1, wherein said device of cell culture chip comprises a buffer layer among the base plate and the cell adhesion layer.

3. The device according to claim 1, wherein said device of base plate is consisted from each of sapphire, SiC, spinel and GaAs group.

4. The device according to claim 1, III-V group nitride film is consisted from one of nitrogen aluminium, nitrogen gallium and nitrogen indium group.

5. The device according to claim 1, III-V group nitride film is grown on the surface of base plate by metalorganic chemical vapor deposition, molecular beam epitaxy and hydride vapor phase epitaxy.

6. The device according to claim 1, each of nerve cell, neural stem cell, artificial neural cell, muscle cell and myocardium cell is culture cell.

7. The device according to claim 2, buffer layer is consisted from each of nitrogen aluminium, and nitrogen gallium.

8. The device according to claim 1, wherein said device may apply to rebuild of disease or nerve damage cause by wounded nerve.

9. The device according to claim 1, wherein said device may combine with a measuring analysis system which is used to measure the cell sensitive of stimulant.

10. A method for culturing cell, wherein providing a place for III-V group nitride to adhere and growth on cell adhesion layer.

11. The method according to claim 10, comprising:
providing a cultivation container;
providing a cell adhesion layer which set in the cultivation container and the cell adhesion layer is one of III-V group nitride compound;
providing a culture put in the cultivation container; and
inoculating a wants cell on surface of the cell adhesion layer.

12. The method according to claim 1, claim III-V group nitride compound is consisted from each of nitrogen aluminium, nitrogen gallium and nitrogen indium group.

13. The method according to claim 1, each of nerve cell, neural stem cell, muscle cell and myocardium cell is culture cell.

14. A device of artificial neural chip combines with a cell culture chip according to claim 10, comprising:
a base plate;
a circuit layer which is formed on the surface of the base plate; and
a cell adhersion layer which is formed on the surface of the circuit layer;
providing a place for cell to adhere and growth
wherein
the cell adhesion layer is a III-V group nitride film.

15. The device according to claim 14, the base plate is base plate of glass or silicon base plate.

16. The device according to claim 15, every sides of this circuit and the cell adhersion layer have numbers of electrode, and numbers of wire link with electrode.

17. The device according to claim 16, wherein the electrode is selected from one of gold, silver, rhenium, ruthenium, rhodium, Pd, silver, osmium and iridium.

18. The device according to claim 16, wherein the wire is comprising from one of indium oxide, tin oxide, cadmium tin oxide, antimony, chromium, silver, copper of the zinc oxide, etc. or metal alloy.

19. The device according to claim 14, III-V group nitride film is consisted from one of nitrogen aluminium, nitrogen gallium and nitrogen indium group.

20. The device according to claim 14, each of nerve cell, artificial neural cell, muscle cell and myocardium cell is culture cell.

21. A device of bio-chip, include:
a cell adhesion layer which provides a place for culture cell to adhere and growth; and a measuring analytical system;
wherein
the cell adhesion layer is a III-V group nitride film.

22. The device according to claim 14, wherein the bio-chip can be medicine sieves, biomedical sensing device and artificial neural network chips.
